(19) United States
(12) Patent Application Publication
(10) Pub. No.: US 2022/0331526 A1
(43) Pub. Date: Oct. 20, 2022

(54) AIR ELIMINATION ASSEMBLIES

(71) Applicant: CareFusion 303, Inc., San Diego, CA (US)

(72) Inventors: Lisa Davis, San Antonio, TX (US); Gregory Hulan, Poway, CA (US); Choi Ting Lau, Santee, CA (US)

(21) Appl. No.: 17/764,504

(22) PCT Filed: Sep. 29, 2020
(86) PCT No.: PCT/US2020/053342

§ 371 Date: Mar. 28, 2022

Related U.S. Application Data
(60) Provisional application No. 62/908,507, filed on Sep. 30, 2019.

(51) Int. Cl. A61M 5/38; A61M 5/142; B01D 19/00; B01D 39/20

(52) U.S. Cl. CPC A61M 5/385; A61M 5/14216; B01D 19/0031; B01D 19/0063; B01D 39/2068; A61M 2005/1652

(57) ABSTRACT

Air elimination assemblies are described herein. An air elimination assembly for eliminating air from a flow of infusate includes a housing and a hydrophobic filter. The housing defines an infusate flow path having an inlet and an outlet, and an air flow path in fluid communication with the infusate flow path and disposed between the inlet and the outlet of the infusate flow path. The hydrophobic filter is disposed in fluid communication with the air flow path, wherein the hydrophobic filter is configured to permit air from the flow of infusate through a hydrophobic filter media and prevent the flow of infusate through the hydrophobic filter media.

### REFERENCE TO RELATED APPLICATIONS

This application is the national stage application pursuant to 35 U. S. C. 371 of International Patent Application No. PCT/US2020/053342, filed on Sep. 29, 2020, which claims priority to U.S. Provisional Patent Application No. 62/908,507, filed Sep. 30, 2019, the entire disclosure of each application being incorporated herein by this reference.

### FIELD OF THE INVENTION

The present disclosure generally relates to systems to direct the flow of medical fluids, and, in particular, systems to eliminate air from medical fluids.

### BACKGROUND

Medical treatments often include the infusion of a medical fluid (e.g., a saline solution, a liquid medication, lipids, blood products, etc.) to patients from a source of fluid, for example, an IV bag or other medical fluid containers. Medical fluids are often delivered by gravity or through the use of an IV pump. In some applications, a silicone pumping segment is utilized with the IV pump. In some applications, a pump cassette is utilized with the IV pump.

During operation, air may be introduced into the IV line from a variety of sources. Sources of air may include the use of porous tubing, a vacuum from the infusate container, outgassing of the medical fluid, improper or insufficient priming of the IV set, and/or ultrasonic agitation of large molecule drugs. IV sets often include an air-in-line sensor or alarm to warn a clinician of an air-in-line condition and/or to stop the infusion.

In some applications, air may be introduced into the IV line, requiring the infusion to be stopped until the air is removed.

### SUMMARY

In some applications, air may be introduced into the IV line during pumping or infusion operations. However, many IV configurations may not remove air-in-line prior to triggering an air-in-line alarm.

Therefore, in some applications, certain IV configurations may unreliably detect and/or remove air-in-line conditions.

The disclosed subject matter relates to air elimination assemblies. In certain embodiments, an air elimination assembly for eliminating air from a flow of infusate is disclosed that comprises a housing defining an infusate flow path having an inlet and an outlet, and an air flow path in fluid communication with the infusate flow path and disposed between the inlet and the outlet of the infusate flow path; and a hydrophobic filter disposed in fluid communication with the air flow path, wherein the hydrophobic filter is configured to permit air from the flow of infusate through a hydrophobic filter media and prevent the flow of infusate through the hydrophobic filter media.

In certain embodiments, an air elimination assembly for eliminating air from a flow of infusate is disclosed that comprises a housing defining a fluid channel having an inlet and an outlet, and an air vent in fluid communication with the fluid channel and disposed between the inlet and the outlet of the fluid channel; and a hydrophobic filter disposed within the fluid channel and covering an entrance of the air vent from the fluid channel, wherein the hydrophobic filter is configured to permit air from the flow of infusate through a hydrophobic filter media into the air vent and prevent the flow of infusate through the hydrophobic filter media.

In certain embodiments, an air elimination assembly for eliminating air from a flow of infusate is disclosed that comprises a housing defining a flow path having a primary input port and an output port, and an air trap path in fluid communication with the flow path between the primary input port and the output port; and a hydrophobic filter in fluid communication with the air trap path wherein the hydrophobic filter is configured to permit air from the flow of infusate through a hydrophobic filter media from the air trap path and prevent the flow of infusate through the hydrophobic filter media.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a perspective view of a filter assembly, in accordance with various aspects of the present disclosure.

FIG. 2 is a cross-sectional view of the filter assembly of FIG. 1, in accordance with various aspects of the present disclosure.

FIG. 3 is a cross-sectional view of a cassette assembly, in accordance with various aspects of the present disclosure.

FIG. 4 is a cross-sectional view of the cassette assembly of FIG. 3 in a first filling phase, in accordance with various aspects of the present disclosure.

FIG. 5 is a cross-sectional view of the cassette assembly of FIG. 3 in an air entry phase, in accordance with various aspects of the present disclosure.

FIG. 6 is a cross-sectional view of the cassette assembly of FIG. 3 in an air evacuation phase, in accordance with various aspects of the present disclosure.

FIG. 7 is a cross-sectional view of the cassette assembly of FIG. 3 in a pumping phase, in accordance with various aspects of the present disclosure.

FIG. 8 is a cross-sectional view of the cassette assembly of FIG. 3 in a second filling phase, in accordance with various aspects of the present disclosure.

FIG. 9 is a cross-sectional view of the cassette assembly of FIG. 3 in a pressure release phase, in accordance with various aspects of the present disclosure.

FIG. 10 is a cross-sectional view of the cassette assembly of FIG. 3 in a third filling phase, in accordance with various aspects of the present disclosure.

FIG. 11 is a cross-sectional view of a cassette assembly, in accordance with various aspects of the present disclosure.

FIG. 12 is a cross-sectional view of a cassette assembly, in accordance with various aspects of the present disclosure.

FIG. 13 is a cross-sectional view of a cassette assembly, in accordance with various aspects of the present disclosure.

### DETAILED DESCRIPTION

The disclosed air elimination assembly incorporates a hydrophobic filter to remove air from infusate flowing through a flow path. The hydrophobic filter can permit air from the flow of infusate through a hydrophobic filter media and prevent the flow of infusate through the hydrophobic filter media. By removing air from the infusate, infusion operations can continue reliably without interruptions and minimizing air-in-line alarms.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to removing air from infusate in a flow path, it is to be understood that this description is only an example of usage and does not limit the scope of the claims. Various aspects of the disclosed air elimination assemblies may be used in any application where it is desirable remove air from a liquid.

The disclosed air elimination assembly overcomes several challenges discovered with respect to certain conventional air elimination assemblies. One challenge with certain conventional air elimination assemblies is that conventional air elimination assemblies may not eliminate air introduced into the IV line, for example, air introduced due to the porosity of a pumping segment. Further, certain conventional air elimination filters may not be positioned prior to air-in-line sensors or alarms due to space constraints and/or the size of certain conventional air elimination filters. Also, certain conventional air elimination filters may utilize small pore size filters (0.2 to 5 microns) and may not effectively be used with large molecule drugs. Further, certain conventional air elimination filters may introduce additional components into the IV set, adding to the cost of the IV set. Also, certain conventional air elimination filters can require an increased priming volume. Because conventional air elimination systems may cause false air-in-line alarms, may not effectively remove air, may not be used with large molecule drugs, introduces additional components, and may require increased priming volumes, the use of conventional air elimination assemblies is undesirable.

Therefore, in accordance with the present disclosure, it is advantageous to provide air elimination assemblies as described herein that effectively eliminate air and reduce air-in-line alarms, while simplifying IV sets, reducing priming volumes, and permitting the elimination of air from large molecule drugs.

An example of an air elimination assembly that effectively eliminates air and reduces air-in-line alarms is now described.

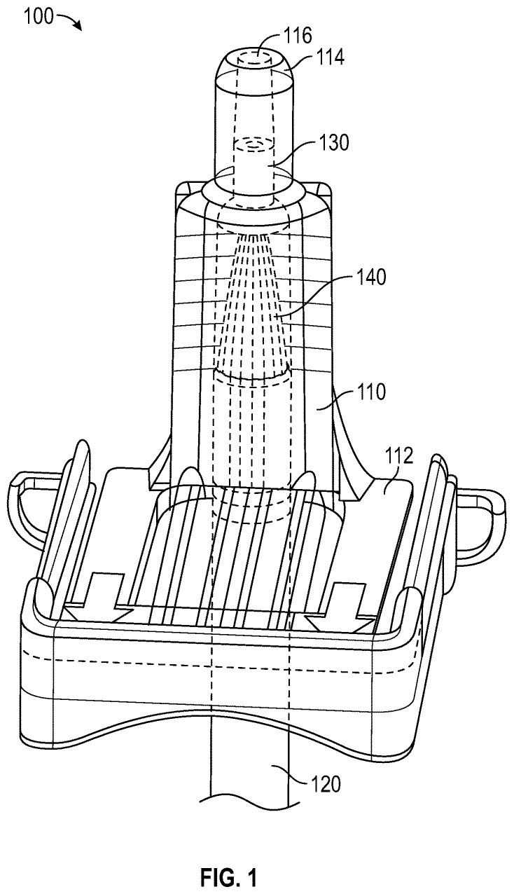

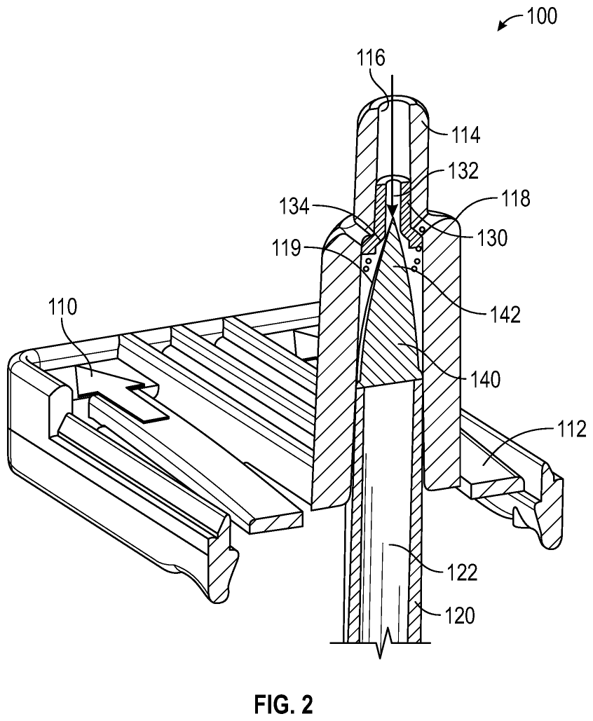

FIG. 1 is a perspective view of a filter assembly 100, in accordance with various aspects of the present disclosure. FIG. 2 is a cross-sectional view of the filter assembly 100 of FIG. 1, in accordance with various aspects of the present disclosure. With reference to FIGS. 1 and 2, the filter assembly 100 removes air from the flow of infusate as it passes through the filter assembly 100.

During operation, infusate flows from the inlet 116 to an outlet 122 through the channel 114 defined within the assembly housing 110. In the depicted example, the inlet 116 receives flow from a pump segment or other suitable infusion pump or container. As illustrated, tubing 120 can be disposed within or engaged with the outlet 122 to receive flow from the channel 114.

In the depicted example, the channel 114 directs the flow of infusate toward the outlet 122. As described herein, the hydrophobic filter 130 disposed within the channel 114 comprises a hydrophobic filter media that prevents the flow of aqueous solutions, such as infusate, therethrough. Therefore, in some embodiments, an infusate channel 132 formed through hydrophobic filter 130 permits the flow of infusate without passing through the hydrophobic filter media.

During operation, infusate flow passes through a hydrophilic filter 140 to prevent the migration of air toward the outlet 122 while allowing infusate to flow from the outlet 122 and to a patient via tubing 120. In the depicted example, the hydrophilic filter 140 is formed from a hydrophilic membrane or filter media that has an affinity for water and therefore adsorbs water or infusate. Hydrophilic filter media can further have a high surface tension value. As a result of the surface chemistry of the hydrophilic filter media, the hydrophilic filter 140 can be wetted by a water film or coating on the surface. Accordingly, an aqueous solution, such as infusate will flow through the hydrophilic filter media while preventing the flow of air therethrough. The hydrophilic filter media can have a pore size ranging from 5 microns to 100 microns or greater, permitting the use of the hydrophilic filter with large molecule drugs. In some applications, hydrophilic filter media can have a greater charge density than hydrophobic filter media.

As illustrated, the base of the hydrophilic filter 140 seals against the inner diameter of the lower fitment or tubing 120 to prevent air from passing into the tubing 120. As can be appreciated, the geometry of the hydrophilic filter 140 can vary to maximize the surface area of the hydrophilic filter media exposed to infusate. Optionally, the hydrophilic filter 140 can include bellows or pleats. In some embodiments, the hydrophilic filter 140 can be tapered, conical, or cylindrically shaped. As illustrated, the hydrophilic filter 140 can at least partially extend into the infusate channel 132 of the hydrophobic filter 130.

In the depicted example, air rejected or prevented from passing through the hydrophilic filter 140 may be trapped within the channel reservoir 119 formed between the hydrophilic filter 140 and the hydrophobic filter 130. During operation, trapped air may remain within the channel reservoir 119 or may pass through the hydrophobic filter media of the hydrophobic filter 130 to be vented into the surroundings via an air vent 118 formed in the assembly housing 110. Optionally, the assembly housing 110 can include any suitable number of air vents 118.

In some embodiments, the hydrophobic filter 130 allows for air to pass through the hydrophobic filter media toward the air vents 118 while preventing infusate from exiting the filter assembly 100 through the air vents 118. In the depicted example, the hydrophobic filter 130 is formed from hydrophobic membranes or filter media that do not adsorb water or infusate, causing water or infusate to bead on the surface instead of adsorbing it. Hydrophobic filter media possess low surface tension values and lack active groups as part of their surface chemistry for the formation of hydrogen bonds with water. Therefore hydrophobic filter media will allow for the passage of air through it, while preventing aqueous solutions such as infusate from entering its pores.

As illustrated, the hydrophobic filter 130 can seal or otherwise cover the air vents 118 to prevent infusate from leaking through the air vents 118. In some embodiments, the hydrophobic filter 130 has a flared profile to engage or seal against the channel 114 adjacent to the air vents 118.

Optionally, the assembly housing 110 can be coupled to the upper fitment of a pump segment, other infusion pump, or to other components in the IV set. In some embodiments, the assembly housing 110 can include a clip 112 to resiliently engage the upper fitment of a pump segment or other components to dispose and integrate the filter assembly 100 with other components of the IV set. The clip 112 can be a biasing member that is configured to retain the assembly housing 110 until it is released by a user. Advantageously, the filter assembly 100 may be disposed before an air-in-line sensor preventing air-in-line alarms.

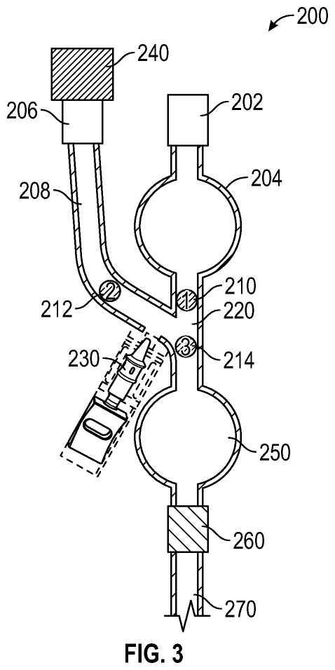

FIG. 3 is a cross-sectional view of a cassette assembly 200, in accordance with various aspects of the present disclosure. In the depicted example, the cassette assembly 200 can facilitate the pumping or delivery of infusate to a patient while eliminating air from the infusate using a cassette pump or other suitable infusion pump. As can be appreciated, similar principles and concepts as described with respect to filter assembly 100 may be utilized with the cassette assembly 200.

As illustrated, the cassette assembly 200 can include a housing that defines an infusate flow path 220 between a primary input port 202 and an output port 270. During operation a pump 230 disposed within the flow path 220 can pump fluid from an infusate source to the patient. In some embodiments, the pump 230 can be a piston pump, or other suitable pump. As can be appreciated, the cassette assembly 200 and the flow path 220 defined therein can be vertically orientated or in any suitable orientation.

As described herein, the cassette assembly 200 can include valves to control the flow of infusate during pumping. For example, the cassette assembly 200 can include an upstream valve 210 disposed upstream of the pump 230 to prevent the backflow of infusate towards the infusate container. Further, the cassette assembly 200 can include a downstream valve 214 to prevent the undesired administration of infusate. As described herein, the upstream valve 210 and the downstream valve 214 can be controlled in sequence with the pump 230 operation. Optionally, the flow path 220 can include pressure domes to facilitate the accumulation and/or equalization of fluid pressure within the flow path. For example, the flow path 220 can include an upstream pressure dome 204 disposed upstream of the upstream valve 210. Similarly, the flow path 220 can include a downstream pressure dome 250 disposed downstream of the downstream valve 214.

In the depicted example, the cassette assembly 200 includes an air trap 208 in fluid communication with the flow path 220 to trap and remove air from the infusate flow. In some embodiments, the air trap 208 is a fluid pathway that allows air to migrate away from the flow path 220. In some embodiments, the air trap 208 can be elevated to allow air to flow up and away from the infusate in the flow path 220. As described herein, the pump 230 can further direct air into the air trap 208.

In some embodiments, the air trap 208 includes a hydrophobic cap or filter 240 disposed over a secondary input port 206 at the end of the air trap 208. In the depicted example, the hydrophobic filter 240 allows for air to be released into the environment without leaking infusate. In some embodiments, the pump 230 can apply positive pressure within the air trap 208 to permit air to be released through the hydrophobic filter 240, reducing air-in-line conditions and related alarm conditions. In some embodiments, the cassette assembly 200 can include an air-in-line sensor 260 to monitor for air within the infusate flow.

Similar to hydrophobic filter 130, the hydrophobic filter 240 can be formed from hydrophobic filter media that permits air to pass through the hydrophobic filter media but prevents the flow of infusate through the hydrophobic filter media. In some embodiments, the hydrophobic filter 240 can be formed of silicon.

Optionally, the hydrophobic filter 240 can be removed enable a secondary concurrent flow through the cassette assembly 200. For example, a secondary fluid source can be coupled to the secondary input port 206 to allow a secondary flow to be introduced into the flow path 220 via the air trap 208.

In some embodiments, an air trap valve 212 disposed within the air trap 208 can control flow to control the flow of air and/or secondary flow through the air trap 208.

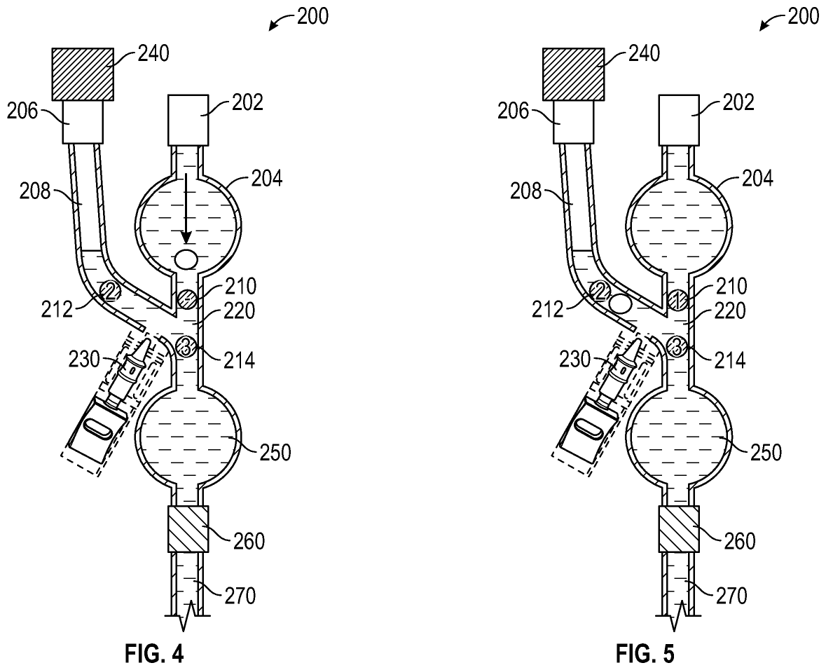

FIGS. 4-10 illustrate various phases of operation of the cassette assembly 200 in certain embodiments. As can be appreciated, various phases of operation can be modified, added, or removed as needed. In some embodiments, the cassette assembly 200 can be primed with infusate prior to operation.

FIG. 4 is a cross-sectional view of the cassette assembly 200 of FIG. 3 in a first filling phase, in accordance with various aspects of the present disclosure. As illustrated, the upstream valve 210 can be moved to an open position, while the pump 230 a pumping piston is retracted, drawing infusate into the flow path 220. As can be appreciated, during a filling phase air may be present in the infusate.

FIG. 5 is a cross-sectional view of the cassette assembly 200 of FIG. 3 in an air entry phase, in accordance with various aspects of the present disclosure. After the filling phase, air trapped within the flow path 220 can migrate toward the air trap 208. In the depicted example, air is trapped within the air trap 208 by the air trap valve 212.

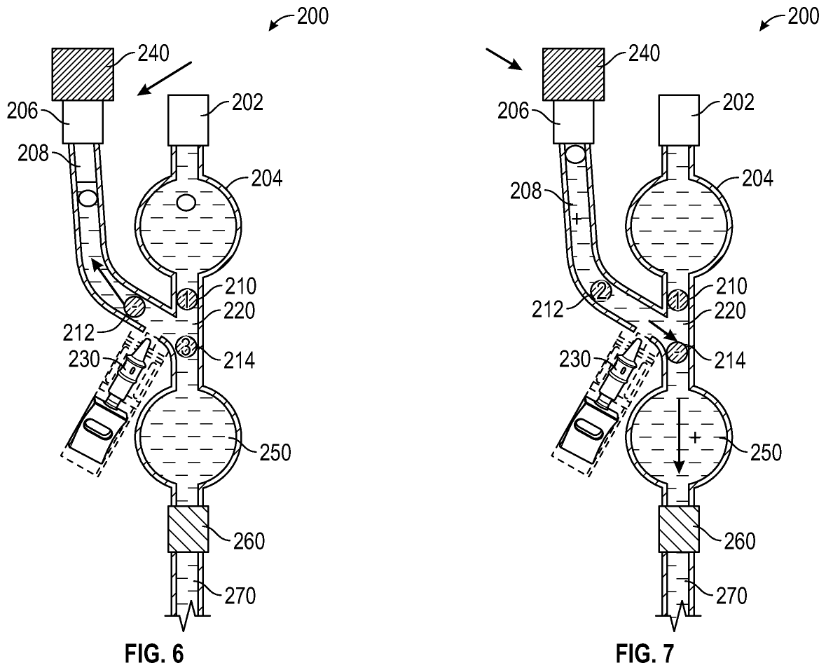

FIG. 6 is a cross-sectional view of the cassette assembly 200 of FIG. 3 in an air evacuation phase, in accordance with various aspects of the present disclosure. In the depicted example, the air trap valve 212 can be moved to an open position, while the pump 230 is actuated, creating positive pressure in the air trap 208. Trapped air within with air trap 208 is moved upward by the pump pressure and buoyancy. As described herein, the trapped air can migrate through the hydrophobic filter 240 while retaining any infusate within the air trap 208.

FIG. 7 is a cross-sectional view of the cassette assembly 200 of FIG. 3 in a pumping phase, in accordance with various aspects of the present disclosure. In the depicted example, the downstream valve 214 is moved to an open position and the pump 230 is actuated creating positive pressure to deliver the infusate toward the patient. As the infusate is pumped toward the output port 270, the infusate may flow through the air-in-line sensor 260. Optionally, positive pressure within the air trap 208 can allow for air to continue to escape through the hydrophobic filter 240.

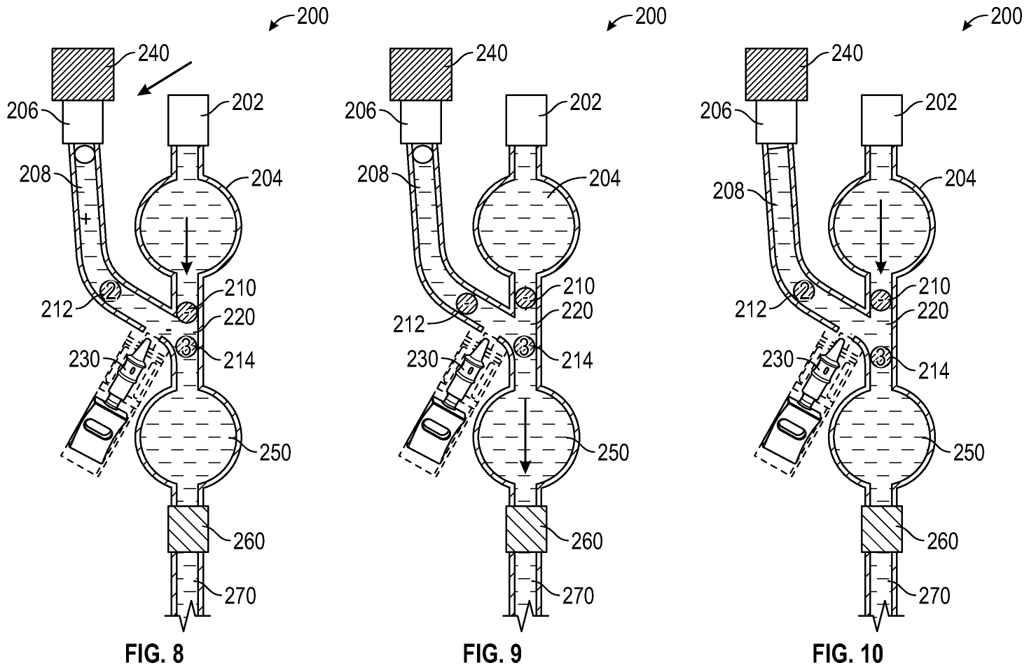

FIG. 8 is a cross-sectional view of the cassette assembly 200 of FIG. 3 in a second filling phase, in accordance with various aspects of the present disclosure. As illustrated, the upstream valve 210 can be moved to an open position, while the pump 230 a pumping piston is retracted, drawing infusate into the flow path 220.

FIG. 9 is a cross-sectional view of the cassette assembly 200 of FIG. 3 in a pressure release phase, in accordance with various aspects of the present disclosure. Optionally, as required, the air trap valve 212 and the upstream valve 210 can be moved to an open position to release pressure within the air trap 208 and the flow path 220.

FIG. 10 is a cross-sectional view of the cassette assembly 200 of FIG. 3 in a third filling phase, in accordance with various aspects of the present disclosure. After the pressure release phase, the cassette assembly 200 can be placed in a filling phase again. As illustrated, the upstream valve 210 can be moved to an open position, while the pump 230 a pumping piston is retracted, drawing infusate into the flow path 220.

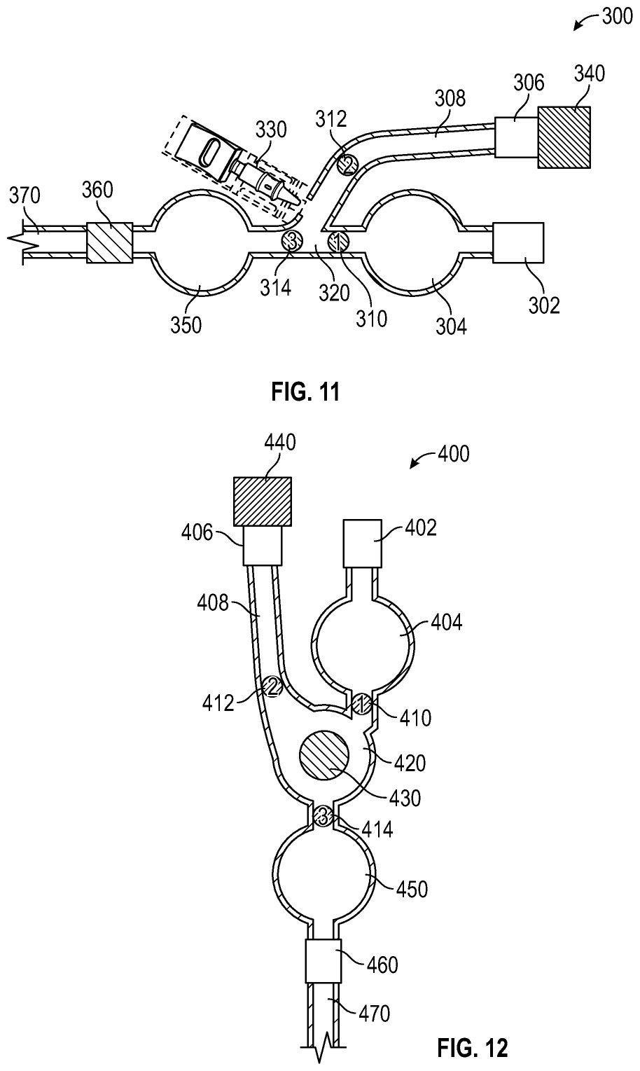

FIG. 11 is a cross-sectional view of a cassette assembly 300, in accordance with various aspects of the present disclosure. In the depicted example, the cassette assembly 300 may include features and elements that are similar to cassette assembly 200 and therefore similar elements may be referred to with similar reference numerals. In some embodiments, the cassette assembly 300 may be horizontally disposed.

FIG. 12 is a cross-sectional view of a cassette assembly 400, in accordance with various aspects of the present disclosure. In the depicted example, the cassette assembly 400 may include features and elements that are similar to cassette assembly 200 and therefore similar elements may be referred to with similar reference numerals. In the depicted example, the cassette assembly 400 can include a silicon pumping segment 430 that is disposed within the flow path 420. During operation, the silicon pumping segment 430 may extend and retract to impart positive and negative pressure as required.

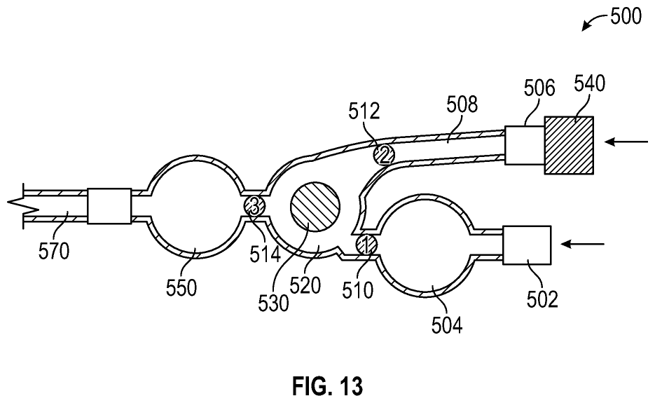

FIG. 13 is a cross-sectional view of a cassette assembly 500, in accordance with various aspects of the present disclosure. In the depicted example, the cassette assembly 500 may include features and elements that are similar to cassette assembly 400 and therefore similar elements may be referred to with similar reference numerals. In some embodiments, the cassette assembly 500 may be horizontally disposed.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An air elimination assembly for eliminating air from a flow of infusate, the air elimination assembly comprising:
a housing defining an infusate flow path having an inlet and an outlet, and an air flow path in fluid communication with the infusate flow path and disposed between the inlet and the outlet of the infusate flow path; and
a hydrophobic filter disposed in fluid communication with the air flow path, wherein the hydrophobic filter is configured to permit air from the flow of infusate through a hydrophobic filter media and prevent the flow of infusate through the hydrophobic filter media.

2. An air elimination assembly for eliminating air from a flow of infusate, the air elimination assembly comprising:
a housing defining a fluid channel having an inlet and an outlet, and an air vent in fluid communication with the fluid channel and disposed between the inlet and the outlet of the fluid channel; and
a hydrophobic filter disposed within the fluid channel and covering an entrance of the air vent from the fluid channel, wherein the hydrophobic filter is configured to permit air from the flow of infusate through a hydrophobic filter media into the air vent and prevent the flow of infusate through the hydrophobic filter media.

3. The air elimination assembly of claim 2, wherein the hydrophobic filter defines an infusate channel through the hydrophobic filter media, permitting the flow of infusate through the fluid channel via the infusate channel.

4. The air elimination assembly of claim 2, wherein the hydrophobic filter comprises a flared portion covering the entrance of the air vent.

5. The air elimination assembly of claim 2, further comprising a hydrophilic filter in fluid communication with the fluid channel, wherein the hydrophilic filter is configured to permit the flow of infusate through a hydrophilic filter media toward the outlet and prevent the flow of air through the hydrophilic filter media.

6. The air elimination assembly of claim 5, wherein the hydrophilic filter comprises a tapered portion.

7. The air elimination assembly of claim 5, wherein the hydrophilic filter is disposed adjacent to the hydrophobic filter within the fluid channel.

8. The air elimination assembly of claim 5, comprising a tubing in fluid communication with the outlet of the fluid channel, wherein the tubing is configured to receive the flow of infusate from the hydrophilic filter.

9. The air elimination assembly of claim 8, wherein a portion of the hydrophilic filter covers a tubing inlet of the tubing.

10. The air elimination assembly of claim 2, wherein the housing comprising a clamping portion.

11. An air elimination assembly for eliminating air from a flow of infusate, the air elimination assembly comprising:
a housing defining a flow path having a primary input port and an output port, and an air trap path in fluid communication with the flow path between the primary input port and the output port; and
a hydrophobic filter in fluid communication with the air trap path wherein the hydrophobic filter is configured to permit air from the flow of infusate through a hydrophobic filter media from the air trap path and prevent the flow of infusate through the hydrophobic filter media.

12. The air elimination assembly of claim 11, wherein the hydrophobic filter comprises silicone.

13. The air elimination assembly of claim 11, wherein the hydrophobic filter is disposed around a secondary input port of the air trap path, the secondary input port disposed away from the flow path.

14. The air elimination assembly of claim 11, further comprising a pump in fluid communication with the flow path and configured to direct air toward the air trap path.

15. The air elimination assembly of claim 14, further comprising an air trap valve disposed within the air trap path and configured to prevent the flow of infusate to the hydrophobic filter in a closed position.

16. The air elimination assembly of claim 14, further comprising an upstream valve disposed within the flow path and configured to prevent the flow of air toward the primary input port in a closed position.

17. The air elimination assembly of claim 14, further comprising a downstream valve disposed within the flow path and configured to prevent the flow of air toward the output port in a closed position.

18. The air elimination assembly of claim 14, wherein the pump comprises a piston pump.

19. The air elimination assembly of claim 14, wherein the pump comprises a silicon pumping segment.

20. The air elimination assembly of claim 11, wherein the flow path includes a pressure dome.
